(19) United States
(12) Patent Application Publication
(10) Pub. No.: US 2026/0041546 A1
(43) Pub. Date: Feb. 12, 2026

(54) SYSTEMS AND METHODS FOR TISSUE RETENTION

(71) Applicants: David S. Rosenberg, West Hollywood, CA (US); Richard A. Thompson, II, Saint Louis Park, MN (US); Jacob T. Wilson, Falcon Heights, MN (US); Mohamed Abdisalan Mohamed, Minneapolis, MN (US)

(72) Inventors: David S. Rosenberg, West Hollywood, CA (US); Richard A. Thompson, II, Saint Louis Park, MN (US); Jacob T. Wilson, Falcon Heights, MN (US); Mohamed Abdisalan Mohamed, Minneapolis, MN (US)

(21) Appl. No.: 19/296,706

(22) Filed: Aug. 11, 2025

Related U.S. Application Data
(60) Provisional application No. 63/682,254, filed on Aug. 12, 2024.

(51) Int. Cl. A61F 2/12; A61B 17/00; A61B 17/08

(52) U.S. Cl. CPC A61F 2/12; A61B 17/08; A61B 2017/00004; A61B 2017/00792; A61B 2017/00796; A61B 2017/081; A61F 2220/0016; A61F 2230/0006; A61F 2250/0037

(57) ABSTRACT

This disclosure describes systems, devices, and methods for tissue retention, for example, tissue retention for aligning tissues around a surgical opening during a surgical procedure. In particular implementations, the systems, devices, and methods described herein can include a tissue retention system that is implantable into a subject to facilitate precise alignment of tissues while surgical treatment areas are closed and additionally maintain the desired alignment of tissues during the healing process. Some systems, devices and methods detailed herein provide tissue retention utilizing a tissue retention system that includes a frame, upper hooks, and lower hooks that can be implanted into a surgical site or wound opening.

### CROSS-REFERENCE TO RELATED APPLICATION

This application claims priority to U.S. Provisional Application No. 63/682,254, filed on Aug. 12, 2024. The disclosure of the prior application is considered part of and is incorporated by reference in the disclosure of this application.

### TECHNICAL FIELD

This disclosure describes systems, devices, and methods for tissue retention, such as tissue retention during and after a surgical procedure. Particular examples described herein provide a tissue retention system that facilitates precise alignment of tissues during surgical procedures, wound closure procedures, and the healing process.

### BACKGROUND

Tissue retention can be utilized in surgical procedures and wound closure procedures to align tissues for closure of surgical openings and wounds. Medical personnel utilize tissue retention to ensure wounds are securely closed before the procedure is complete. Improved aesthetic outcomes including reduced scarring and desirable tissue shape involve added procedure time for medical personnel. During healing and after the procedure, the aesthetics can change and worsen as the patient heals. The aesthetic changes result in a reduction in patient aesthetics and a worsen patient outcomes that can involve subsequent medical procedures to repair the undesirable aesthetics.

### SUMMARY

This disclosure describes systems, devices, and methods for tissue retention, for example, tissue retention for aligning tissues around a surgical opening during a surgical procedure. In particular implementations, the systems, devices, and methods described herein can include a tissue retention system that is implantable into a subject to facilitate precise alignment of tissues while surgical treatment areas are closed and additionally maintain the desired alignment of tissues during the healing process. In some examples detailed below, a tissue retention system can include hooks that anchor into a dermal layer of tissue to thereby reduce or eliminate the surgical time traditionally consumed by dermal stitches or staples during the closing of a surgical opening. The tissue retention system also includes hooks that connect to an epidermis layer of tissue to facilitate precise alignment (e.g., centered at an anatomic site even after bandaging for some implementations) and shaping (e.g., a circular shape in some implementations) of the surgical treatment area for closing in a manner that achieves desirable aesthetics after the surgical opening is closed and throughout the healing process. Additionally, the system can include a bioresorbable material that can be implanted in the patient to facilitate desirable alignment throughout the healing process without a follow up procedure to remove the system.

Among other benefits, some systems and methods described herein can advantageously provide a more efficient and precise approach to closing surgical openings of a patient that efficiently closes the surgical opening and precisely aligns tissues to facilitate improved patient healing and aesthetic outcomes. Additionally, some embodiments described in more detail below can achieve an efficient, reliable solution that provides increased operation speed and improved reliability and precision of the closed surgical opening that facilitate repeatable and desirable alignment throughout the healing process. This improved reliability and precision is advantageously achieved without a follow up procedure.

Some embodiments described herein include a tissue retention system. The tissue retention system can include a frame, upper hooks extending from the frame that include a plurality of radially inward arcuate hooks such that a free end of each of the upper hooks is oriented directionally inward towards a central axis of the tissue retention system, and lower hooks extending from the frame that include a plurality of radially outward arcuate hooks such that a free end of each of the lower hooks is oriented directionally outward away from the central axis of the tissue retention system.

Such a system can include one or more of the following optional features. The tissue retention system can include a bioresorbable material. The bioresorbable material is Poly lactic-co-glycolic acid (PLGA). The frame is a ring. The free end of each of the upper hooks is positioned above a longitudinal axis of the tissue retention system. The free end of each of the lower hooks is positioned below a longitudinal axis of the tissue retention system. An upper hook base of each upper hook connects each upper hook to a lateral surface of the frame. A lower hook base of each lower hook connects each lower hook to a bottom surface of the frame. The upper hooks have an upper hook height and the lower hooks have a lower hook height. The upper hook height and the lower hook height are about equal. The upper hook height is smaller than the lower hook height. The system can include a 2:1 ratio of upper hooks to lower hooks. The frame has an inner diameter between 1.6 and 2 inches. The frame has an outer diameter between 1.8 and 2.2 inches. The system can include a system height that extends vertically between the free end of the upper hooks and the free end of the lower hooks. The system height is between 0.2 and 0.7 inches. The tissue retention system is configured for implantation in a breast of a subject. The frame is configured to extend around a nipple of the breast. The lower hooks are configured to anchor into a dermal layer of tissue of the breast. The upper hooks are configured to connect to an epidermis layer of tissue surrounding an areola of the breast.

Some embodiments described herein include a method of installing a tissue retention system. The method can include preparing an installation site that includes a surgical opening in at least an epidermis layer of tissue of a breast of a subject, aligning the tissue retention system around an areola of the breast, inserting lower hooks of the tissue retention system into a dermal layer of tissue surrounding the areola, positioning portions of the epidermis layer that surround the areola onto upper hooks of the tissue retention system, and closing the epidermis layer by attaching the epidermis layer around a circumference of the areola between the upper hooks and an outer edge of the areola, thereby closing the surgical site with the tissue retention system implanted in the breast.

Such a method can include one or more of the following optional features. Closing the epidermis layer can include installing stitches through the epidermis layer surrounding the areola. The upper hooks hold the epidermis layer in position during the closing of the epidermis layer. The method includes adjusting portions of the epidermis layer onto the upper hooks after positioning portions of the epidermis onto the upper hooks and before closing the epidermis layer. The adjusting includes shaping an interface between the epidermis layer and the outer edge of the areola to generate a round shape. The tissue retention system biodegrades within the breast over a period of time after the surgical site is closed. The tissue retention system remains in position during the period of time. The tissue retention system comprises a bioresorbable material. The bioresorbable material is Poly lactic-co-glycolic acid (PLGA). The upper hooks and the lower hooks extend from a frame of the tissue retention system. The upper hooks comprise a plurality of radially inward arcuate hooks such that a free end of each of the upper hooks is oriented directionally inward towards a central axis of the tissue retention system. The lower hooks include a plurality of radially outward arcuate hooks such that a free end of each of the lower hooks is oriented directionally outward away from the central axis of the tissue retention system. After the surgical site is closed, the tissue retention system is positioned posterior to the epidermis layer. After the surgical site is closed, the upper hooks are positioned anterior to the lower hooks.

Some embodiments described herein include a tissue retention system for breast reconstruction. The system includes a frame, upper engagement tines extending from the frame that have free ends oriented directionally inward towards a central axis of the tissue retention system, and lower engagement tines extending from the frame that have free ends oriented directionally outward away from the central axis of the tissue retention system.

Some embodiments described herein include a tissue retention system for cosmetic surgery. The system includes a frame, upper tissue graspers extending from the frame that include pointed free ends, and lower tissue graspers extending from the frame that include pointed free ends.

Some embodiments described herein include a tissue retention system for abdominal cosmetic surgery. The system includes a frame, upper hooks extending from the frame that include a plurality of radially inward arcuate hooks such that a free end of each of the upper hooks is oriented directionally inward towards a central axis of the tissue retention system, and lower hooks extending from the frame that include a plurality of radially outward arcuate hooks such that a free end of each of the lower hooks is oriented directionally outward away from the central axis of the tissue retention system.

Some embodiments described herein include a tissue retention system. The tissue retention system includes an upper frame that has upper hooks extending from the upper frame that include a plurality of radially inward arcuate hooks such that a free end of each of the upper hooks is oriented directionally inward towards a central axis of the tissue retention system, and a lower frame that has lower hooks extending from the lower frame that include a plurality of radially outward arcuate hooks such that a free end of each of the lower hooks is oriented directionally outward away from the central axis of the tissue retention system.

Some embodiments described herein include a tissue retention system. The system includes an upper frame that has upper hooks extending from the upper frame that include a plurality of radially inward arcuate hooks such that a free end of each of the upper hooks is oriented directionally inward towards a central axis of the tissue retention system, and a lower frame that has lower hooks extending from the lower frame.

Some embodiments described herein include a tissue retention system. The system includes an upper frame that has upper loops extending from the upper frame, and a lower frame that has lower hooks extending from the lower frame.

Some embodiments described herein include a tissue retention system. The system includes an upper frame that has a plurality of receptacles, and a lower frame that has a plurality of posts and lower hooks extending from the lower frame. The plurality of receptacles are configured to receive the plurality of posts to connect the upper frame and the lower frame to each other.

Particular implementations can, in certain instances, realize one or more of the following advantages. First, some embodiments described herein provide a more efficient and precise approach to closing surgical openings of a patient that effectively closes the surgical opening with a reduced surgical procedure time. Second, particular solutions described herein advantageously facilitate precise alignment of tissues and improve patient aesthetic outcomes including improvements in the shape and post-surgery cosmetic appearance of the treatment area. Third, some embodiments described herein provide a bioresorbable implant that maintains the shape and physician-selected position of the implant and healed tissue at the treatment area even after bandaging, rebandaging, and multi-day healing. Fourth, some embodiments described herein advantageously provide a solution that does not involve a subsequent procedure for removal and facilitates improved patient outcomes. Fifth, some embodiments described herein provide precise alignment of tissues without the intervention of additional surgical tools and with a reduced amount of dermal suturing or staples at the treatment area.

The details of one or more implementations are set forth in the accompanying drawings and the description below. Other features, objects, and advantages will be apparent from the description and drawings, and from the claims.

### DESCRIPTION OF DRAWINGS

FIG. 1 shows a perspective view of an example system for tissue retention, consistent with some embodiments of this disclosure.

FIG. 2 shows a perspective view of the tissue retention system of FIG. 1.

FIG. 3 shows a top view of the tissue retention system of FIG. 1.

FIG. 4 shows a side view of the tissue retention system of FIG. 1.

FIG. 5 shows the tissue retention system of FIG. 1 at a step of an installation method prior to installation in a subject, consistent with some embodiments of this disclosure.

FIG. 6 shows the tissue retention system of FIG. 1 at a step of the installation method with the lower hooks inserted into the tissue of the breast of the subject, consistent with some embodiments of this disclosure.

FIG. 7 shows a cross section of the breast with the tissue retention system installed at the installation step of FIG. 6.

FIG. 8 shows the tissue retention system of FIG. 1 at a step of the installation method with portions of the epidermis layer positioned onto upper hooks of the tissue retention system.

FIG. 9 shows the tissue retention system of FIG. 9 with additional portions of the epidermis layer positioned onto upper hooks of the tissue retention system.

FIG. 10 shows the tissue retention system of FIG. 9 at a step of the installation method adjustments made to portions of the epidermis layer positioned onto upper hooks of the tissue retention system.

FIG. 11 shows retention system of FIG. 1 at a step of the installation method where the epidermis layer is closed around a circumference of the areola.

FIG. 12 shows a cross section of the breast with the tissue retention system installed at the installation step of FIG. 11.

FIG. 13 shows an example tissue retention system prior to installation in an abdomen of a subject, consistent with embodiments of the present disclosure.

FIG. 14 shows a perspective view of an example tissue retention system, consistent with embodiments of the present disclosure.

FIG. 15 shows a side view of the tissue retention system of FIG. 14.

FIG. 16 shows a top perspective view of an example tissue retention system, consistent with embodiments of the present disclosure.

FIG. 17 shows a bottom perspective view of an example tissue retention system, consistent with embodiments of the present disclosure.

FIG. 18 shows a top perspective view of an example tissue retention system, consistent with embodiments of the present disclosure.

FIG. 19 shows a bottom perspective view of an example tissue retention system, consistent with embodiments of the present disclosure.

FIG. 20 shows a top perspective view of an example tissue retention system, consistent with embodiments of the present disclosure.

FIG. 21 shows an exploded perspective view of an example tissue retention system, consistent with embodiments of the present disclosure.

Like reference symbols in the various drawings indicate like elements.

### DETAILED DESCRIPTION

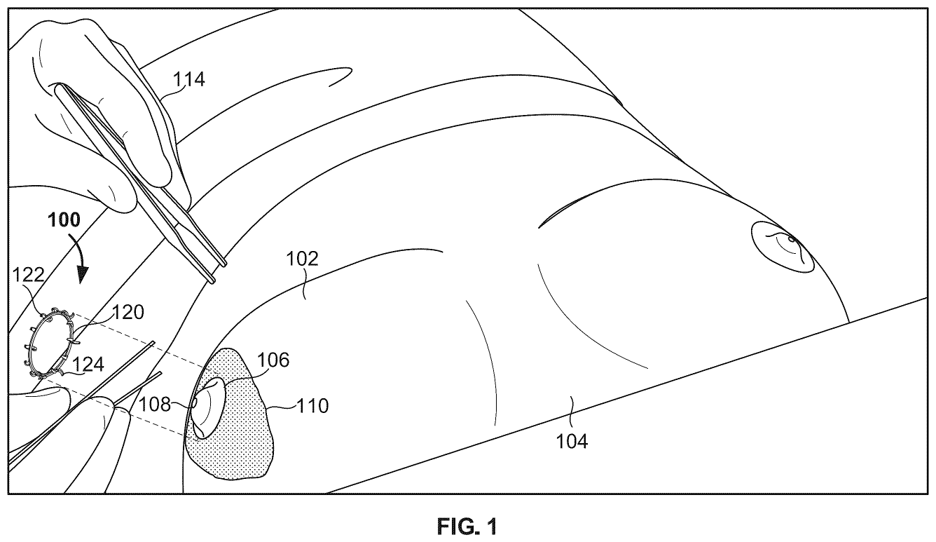

Referring to FIG. 1, some embodiments of a tissue retention system 100 can be implemented to align and retain tissues of a patient in position while the surgical opening is closed and during the subsequent healing process. In the depicted embodiment, the system 100 comprises an implant structure that is implantable into a breast 102 of a patient 104. In the depicted embodiment, the system 100 can be implanted around an areola 106 and a nipple 108, for example, to realign and retain skin toward the areola during a breast surgery. As detailed below, the system 100 facilitates the precise positioning of tissues around the areola during the closing of a surgical opening 110. The system 100 can be a ring-shaped device that includes a frame 120, upper hooks 122, and lower hooks 124. The system 100 can include bioresorbable materials that facilitate implantation of the system 100 that the patient will absorb over time without a subsequent procedure to remove the system 100 after the surgical opening 110 is closed.

As described in more detail below, the system 100 is configured to be aligned around the areola 106 and inserted into the breast 102 by a user 114 (e.g., a surgeon or other medical personnel). During the initial insertion, the lower hooks 124 are configured to anchor into one or both of the dermis and subcutaneous layer of the breast 102 surrounding the areola 106 to facilitate subsequent positioning of the epidermis layer of tissue onto the upper hooks 122. In some embodiments, the tissue retention system 100 can be implemented in one or more medical procedures that include the closing of surgical or wound openings. For example, the system 100 can be implanted in the breast 102 during a cosmetic surgery procedure such as a mastopexy, breast reconstruction, breast augmentation, breast reduction, mastectomy, lumpectomy, among others. In some implementations, the system 100 can be implanted in other areas such as an abdomen of a patient during an abdominoplasty, among other procedures.

In this embodiment, the system 100 is illustrated vertically above an insertion site within the surgical opening 110 prior to the user 114 installing the system 100. The system 100 can be installed by hand, as will be described in further detail below. The user 114 can hold the system 100 to align the system around the circumference of the areola 106 (e.g., centered around the areola 106) and insert the lower hooks 124 into a dermal layer of tissue. In the illustrated embodiment, implantation of the system 100 facilitates alignment of the tissues surrounding the areola 106 and improves aesthetic outcomes for the patient 104. The aesthetic outcomes can include improved shaping during a cosmetic surgery procedure such as a mastopexy, breast reconstruction, breast augmentation, breast reduction, among others. The improved shaping can facilitate improved uniformity of the shape of the areola 106 after healing, enhancing a rounded shape that is maintained throughout the healing process. This uniformity can advantageously provide better shape matching during symmetrical cosmetic procedures such as when both breasts undergo procedures.

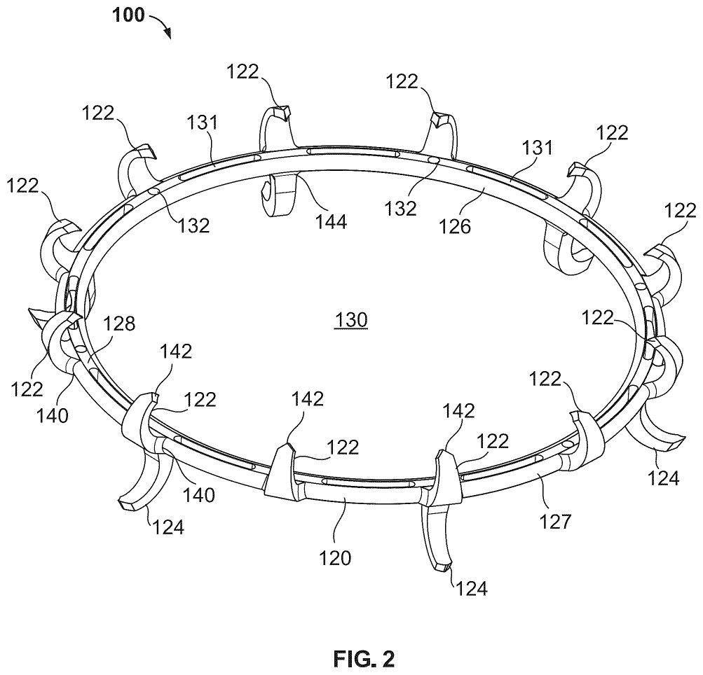

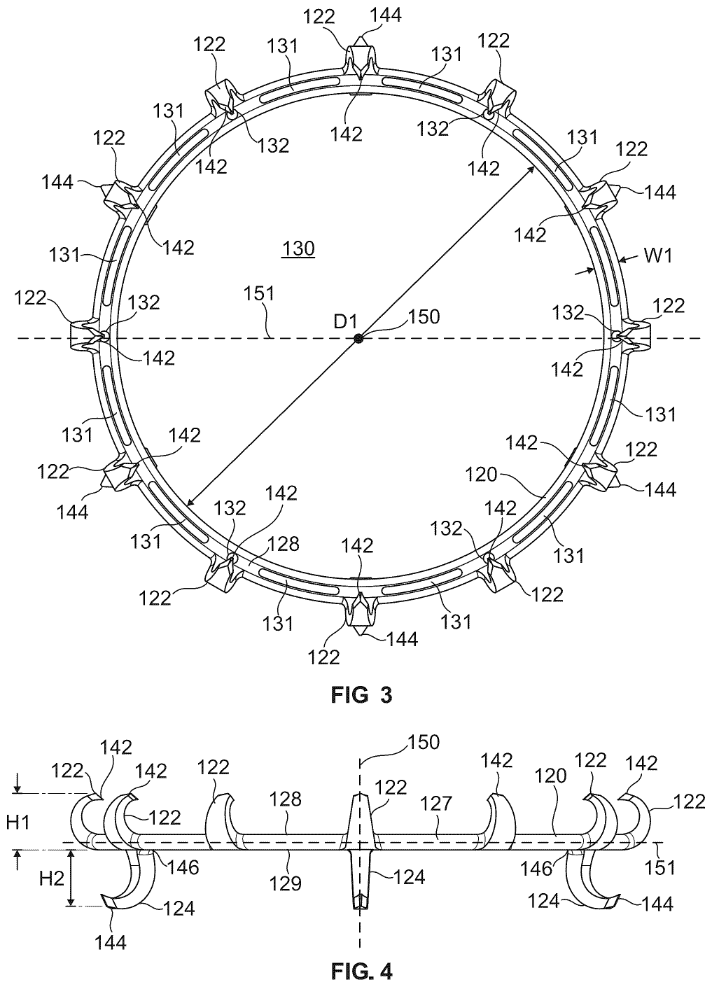

Referring to FIGS. 2-4, the tissue retention system 100 includes the frame 120 and includes upper hooks 122 and lower hooks 124 that extend from the frame 120. The frame 120 can be a rounded shape such as a circle, an oval, an ellipse, or other shapes that extend around an internal opening 130 of the system 100. In some implementations, the internal opening 130 is configured to extend around an areola and a nipple (e.g., areola 106 and nipple 108) to position the system 100 around an areola of a subject.

The frame 120 can be a ring that extends around the internal opening 130. In some embodiments, the frame 120 defines a round shape that facilitates shaping of a surgical opening around an areola of a subject to advantageously secure tissues in a round shape. The shape of the frame 120 can define a shape for forming and aligning tissues to facilitate desirable aesthetics for the patient after healing is complete.

The frame 120 can include an inner surface 126, a lateral surface 127, a top surface 128, and a bottom surface 129. In some embodiments, the inner surface 126 and the lateral surface 127 can be rounded surfaces. The top surface 128 and the bottom surface 129 can be flat surfaces or generally flat surfaces. In some embodiments, the top surface 128 and the bottom surface 129 can be oriented parallel to each other.

The frame 120 can include a plurality of openings spaced apart around the frame 120. In some embodiments, the openings include different shapes including elongate openings 131 and round openings 132. The plurality of openings (e.g., including the elongate openings 131 and round openings 132) can extend through the frame 120, through both the top surface 128 and the bottom surface 129. In some embodiments, the elongate openings 131 can extend between the upper hooks 122. For example, an elongate opening 131 can be positioned between each of the upper hooks 122. The round openings 132 can be positioned along the frame 120 at various positions and can be aligned with a connection of one or more of the upper hooks 122 to the frame. For example, round openings 132 can be aligned with every other upper hook 122. In some embodiments, the plurality of openings includes a pattern of openings including two consecutive elongate openings 131 separated by a single round opening 132. The shape and arrangement of the openings (e.g., elongate openings 131 and round openings 132) can vary. The plurality of openings in the frame 120 can provide a reduction in the material and weight of the system 100. The reduction in material can also increase a surface area of the frame 120 and can advantageously facilitate bio-resorption of the system 100 by the patient. Specifically, the speed of bio-resorption of the system 100 can be increased with the increased surface area in implementations where the system 100 includes bioresorbable materials.

In some embodiments, the frame 120 includes an inner diameter D1. The inner diameter D1 extends from the inner surface 126 across the internal opening 130 to the inner surface 126 on an opposite side of the frame 120. The inner diameter D1 can be between 1 and 3 inches, between 1.5 and 2.5 inches, between 1.6 and 2 inches, between 1.7 and 1.9 inches, between 1.8 and 1.9 inches, or 1.86 inches.

In some embodiments, the frame 120 includes a frame width W1. The frame width W1 extends from the inner surface 126 to the lateral surface 127. The frame width can be between 0.05 and 0.3 inches, between 0.05 and 0.2 inches, between 0.07 and 0.15 inches, between 0.08 and 0.12 inches, or 0.1 inches.

The frame 120 can include an outer diameter that is a combination of the inner diameter D1 and the frame width W1. The outer diameter can be between 1 and 4 inches, 1.5 and 3.5 inches, 1.05 and 3.3 inches, between 1.55 and 2.7 inches, between 1.5 and 2.5 inches, between 1.6 and 2.3 inches, between 1.67 and 2.15 inches, between 1.78 and 2.02 inches, 1.9 inches, 2 inches, or 1.96 inches. The system 100 includes the upper hooks 122 that extend from the

frame 120. In some embodiments, each of the upper hooks 122 can include an upper hook base 140 that connects each upper hook 122 to the lateral surface 127 of the frame 120. The upper hooks 122 can be a plurality of radially inward arcuate hooks. The upper hooks 122 can be a plurality of upper hooks that are evenly spaced apart around the frame 120. In the illustrated embodiment, the system 100 includes twelve upper hooks 122. Some embodiments can include four, five, six, seven, eight, nine, ten, eleven, twelve, thirteen, fourteen, fifteen, sixteen, seventeen, eighteen, nineteen, twenty, or more upper hooks 122.

In some embodiments, the upper hooks 122 initially extend laterally outward from the lateral surface 127. The arcuate shape of the upper hooks 122 directs each of the free ends 142 towards a central axis 150 of the tissue retention system 100. For example, a free end 142 of each of the upper hooks is oriented directionally inward towards the central axis 150. The free end 142 of each of the upper hooks 122 can be spaced apart from the frame 120 (e.g., positioned above the frame 120 when the frame is gravitationally below the upper hooks 122). Each of the free ends 142 can be aligned with the frame 120 such that the upper hooks 122 do not extend interior to the inner surface 126 of the frame 120. For example, each of the free ends 142 can be aligned vertically above the frame 120 such that the free ends 142 are positioned over the frame 120 when the frame 120 is gravitationally below the free ends 142.

The free end 142 of each of the upper hooks 122 can be a sharpened end or pointed end that is configured to engage with an epidermis layer of tissue when the tissue retention system 100 is installed in a patient. In some implementations, the free end 142 is pointed to engage with a posterior side of an epidermis layer of tissue without penetrating through to an anterior side of an epidermis layer of tissue.

The free ends 142 of the upper hooks 122 can be spaced apart from the frame 120 at an upper hook height H1. The upper hook height H1 can extend from the bottom surface 129 of the frame 120 to the free ends 142 of each hook. For example, the upper hook height H1 can position each of the free ends 142 of the upper hooks 122 gravitationally above a longitudinal axis 151 of the system 100 when the frame 120 is gravitationally below the free ends 142. In some embodiments, the upper hook height H1 is the same for each of the upper hooks 122. The upper hook height H1 can be between 0.1 and 0.5 inches, between 0.15 and 0.40 inches, between 0.2 and 0.3 inches, or 0.22 inches.

The system 100 includes the lower hooks 124 that extend from the frame 120. In some embodiments, each of the lower hooks 124 can include a lower hook base 146 that connects each lower hook 124 to the bottom surface 129 of the frame 120. The lower hooks 124 can be a plurality of radially outward arcuate hooks. The lower hooks 124 can be a plurality of lower hooks that are evenly spaced apart around the frame 120. In the illustrated embodiment, the system 100 includes six lower hooks 124. Some embodiments can include one, two, three, four, five, six, seven, eight, nine, ten, eleven, twelve, thirteen, fourteen, fifteen, sixteen, seventeen, eighteen, nineteen, twenty, or more lower hooks 124. In some embodiments, the system 100 includes a 2:1 ratio of upper hooks 122 to lower hooks 124. For example, the system 100 can include a pattern of upper hooks 122 and lower hooks 124 where every-other upper hook 122 has a lower hook 124 aligned with the upper hook 122. In some embodiments, the pattern of upper hooks 122 and lower hooks 124 can vary. For example, the upper hooks 122 and lower hooks 124 can be each be positioned anywhere along the frame 120.

In some embodiments, the lower hooks 124 initially extend downwardly away from the bottom surface 129. The arcuate shape of the lower hooks 124 directs each of the free ends 144 away from the central axis 150 of the tissue retention system 100. For example, the free end 144 of each of the lower hooks 124 is oriented directionally outward away from the central axis 150. The free end 144 of each of the lower hooks 124 can be spaced apart from the frame 120 (e.g., positioned below the frame 120 when the frame is gravitationally above the lower hooks 124). Each of the free ends 144 of the lower hooks can extend laterally from the lateral surface 127 of the frame 120 such that the free ends 144 of the lower hooks 124 can be positioned outside of the frame 120.

The free end 144 of each of the lower hooks 124 can be a sharpened end or pointed end that is configured to engage with a dermal layer of tissue when the tissue retention system 100 is installed in a patient. In some implementations, the free end 144 is pointed to engage within the dermal layer of tissue to anchor the system 100 in position within the patient. The lower hooks 124 can be installed completely in the dermal layer of tissue while the upper hooks 122 remain exposed for connection of the upper hooks 122 to a posterior side of an epidermis layer of tissue without penetrating through to an anterior side of an epidermis layer of tissue.

The free ends 144 of the lower hooks 124 can be spaced apart from the frame 120 at a lower hook height H2. The lower hook height H2 can extend from the bottom surface 129 of the frame 120 to the free ends 144 of each lower hook 124. In some embodiments, the upper hook height H2 is the same for each of the lower hooks 122. The upper hook height H2 can be between 0.1 and 0.5 inches, between 0.15 and 0.40 inches, between 0.2 and 0.3 inches, or 0.22 inches. In some embodiments, the upper hook height H1 and the lower hook height H2 are equal or about the same. In some embodiments, the upper hook height H1 is smaller than the lower hook height H2. In some embodiments, the upper hook height H1 is larger than the lower hook height H2.

In some embodiments, the system 100 includes a system height that extends from the free ends 144 of the lower hooks 124 to the free ends 142 of the upper hooks 122. In some implementations, the system height can be a combination of the upper hook height H1 and the lower hook height H2. The system height can be between 0.1 and 1.5 inches, between 0.1 and 1.0 inches, between 0.2 and 0.7 inches, between 0.2 and 0.6 inches, between 0.3 and 0.6 inches, between 0.4 and 0.5 inches, or 0.44 inches.

In some embodiments, the system 100 can include tissue graspers (e.g., in addition to or instead of the upper hooks 122 and the lower hooks 124). The tissue graspers can include upper tissue graspers and lower tissue graspers that each extend from the frame 120. The tissue graspers can each include a pointed free end to engage with tissues (e.g., in a similar manner as upper hooks 122 and lower hooks 124). In some embodiments, the system 100 can include engagement tines such as hooks, barbs, anchors, and preferably in the form of hooks for the depicted embodiment. The engagement tines can include upper engagement tines and lower engagement tines that each extend from the frame 120. The engagement tines can each include a pointed free end to engage with tissues (e.g., in a similar manner as upper hooks 122 and lower hooks 124).

In some implementations, the system 100 can have a variety of sizes that can be selected for different sized treatment areas. For example, patients undergoing breast reconstruction or mastopexy procedures can have different sized breasts, nipples, or areolas. The system 100 can have different sizes, including different diameters (e.g., larger or smaller than D1), different upper hook heights (e.g., larger or smaller than upper hook height H1), different lower hook heights (e.g., larger or smaller than lower hook height H2), and different system heights.

In some embodiments, the system 100 includes a bioresorbable material. For example, the system 100 can include a single bioresorbable material or combinations of bioresorbable materials. The system 100 can be implanted in a patient, and, over time, the patient's body can absorb the system 100. After implantation, the system 100 can gradually biodegrade and naturally absorb into the patient's body without a follow up procedure to remove the system 100 from the patient. For example, the system 100 can biodegrade and absorb into the patient's body within one week, two weeks, three weeks, four weeks, five weeks, six weeks, seven weeks, eight weeks, nine weeks, ten weeks, or more. In some embodiments, the system 100 includes at least one bioresorbable material such as Poly lactic-co-glycolic acid (PLGA), polyglycolic acid (PGA), poly-l-lactic acid (PLLA), poly-d-lactic acid, polycyanoacrylates, polyanhydrides, polypropylene fumarate, or combinations thereof. The system 100 can be manufactured with the bioresorbable material by injection molding, molding, additive manufacturing, or other techniques.

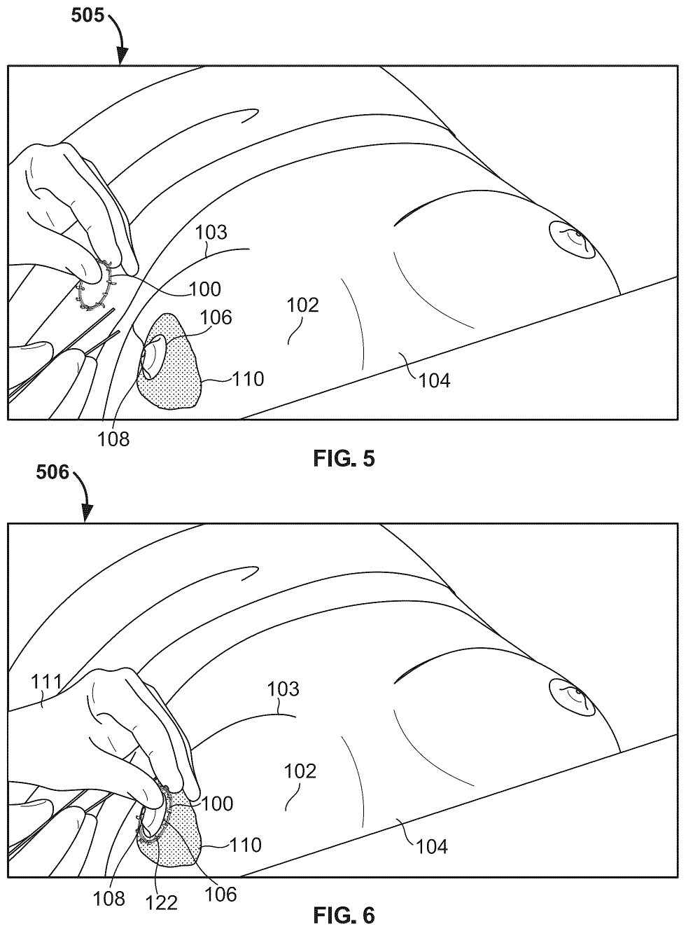

Referring to FIG. 5, the tissue retention system 100 is at a step 505 of an installation method 500 prior to installation of the system 100 in the subject 104. The step 505 can include preparation of the surgical opening 110 and the surgical site. The system 100 can be installed in the subject 104 during a cosmetic surgery procedure such as a mastopexy as illustrated. The mastopexy procedure can include the creation of the surgical opening 110. In some implementations, the surgical opening 110 can be created using a lollipop incision where the incision 503 extends through an epidermis layer of tissue from a base 103 of the breast 102 and extends around the areola 106. In some implementations, the procedure can utilize an anchor incision, a lollipop incision, a donut incision, or others. For example, the anchor incision can extend along the base 103 of the breast 102 and extend from the base 103 and around the areola 106. A donut incision can include an incision that extends around the areola 106. In some implementations, the donut incision does not extend to the base 103 of the breast 102.

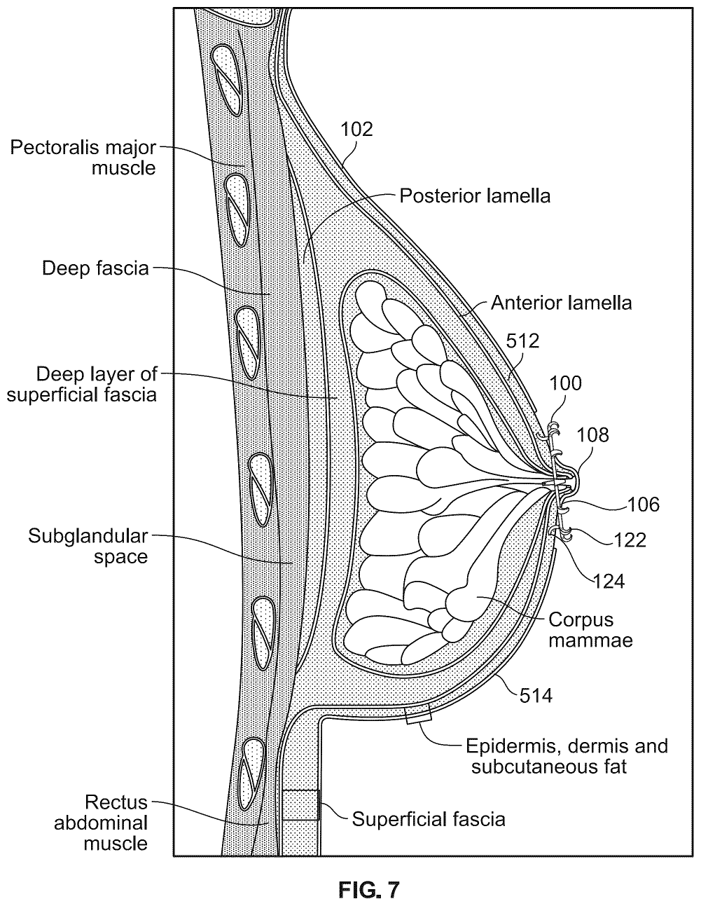

FIG. 6 shows the tissue retention system 100 at a step 506 of the installation method 500 with the lower hooks 124 inserted into the tissue of the breast 102 of the subject 104. The lower hooks 124 are not visible in FIG. 6 and are inserted by the user 111 (e.g., surgeon, doctor, nurse, or other medical personnel) into a dermal layer of the patient (see e.g., dermal layer 517 of FIG. 7). For example, the user 111 can hold and insert the system 100 by hand without the intervention of additional surgical tools. Prior to and during the insertion of the system 100, the user 111 aligns the system 100 around the areola 106 of the breast 102. For example, the user 111 can align the system 100 around the areola 106 and the nipple 108 such that the system 100, when installed is centered around the areola 106.

FIG. 7 shows a cross section of the breast 102 with the tissue retention 100 system installed at the step 506 of FIG. 6. The system 100 is installed around the areola 106 with the lower hooks 124 installed in a dermal layer 512 (e.g., in the dermis, the subcutaneous layer along the underside of the dermis, or both), which thereby permits the patient's epidermis layer 514 to be adjusted toward the upper hooks 122 as described below. The upper hooks 122 remain exposed at step 506 and extend outwardly from the lower hooks 124 that anchor the system 100 into the dermal layer 512 surrounding the areola 106.

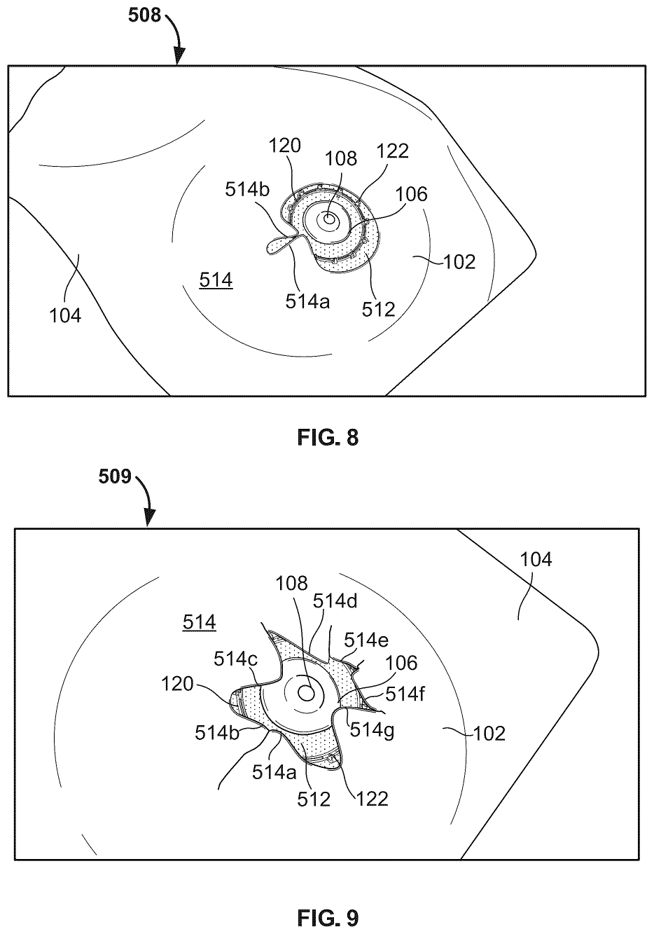

FIG. 8 shows the tissue retention system 100 at a step 508 of the installation method with portions of the epidermis layer 514 positioned onto the upper hooks 122 of the tissue retention system 100. For example, portions 514a and 514b of the epidermis layer 514 can be positioned onto one or more of the upper hooks 122. The user can position the portions 514a and 514b onto the upper hooks 122, and the upper hooks 122 can engage with a posterior side of the epidermis layer 514 to hold the portions 514a, 514b in the position chosen by the user. The user can connect various portions in addition the portion 514a, 514b of the epidermis around the areola 106 to shape the tissues around the areola before securely closing the surgical opening.

FIG. 9 shows the tissue retention system 100 at step 509 of FIG. 9 with additional portions 514c-g of the epidermis layer 514 positioned onto the upper hooks 122 of the tissue retention system 100. In some embodiments, the user can connect various portions (e.g., portions 514a-g) of the epidermis 514 to the upper hooks 122 surrounding the areola. The upper hooks 122 can hold the epidermis in position without stitches, staples, or other attachment mechanisms. The upper hooks 122 can facilitate an adjustment of one or more of the portions 514a-g of the epidermis 514 so that the user can shape the tissues around the areola 106 before completing the closure of the opening.

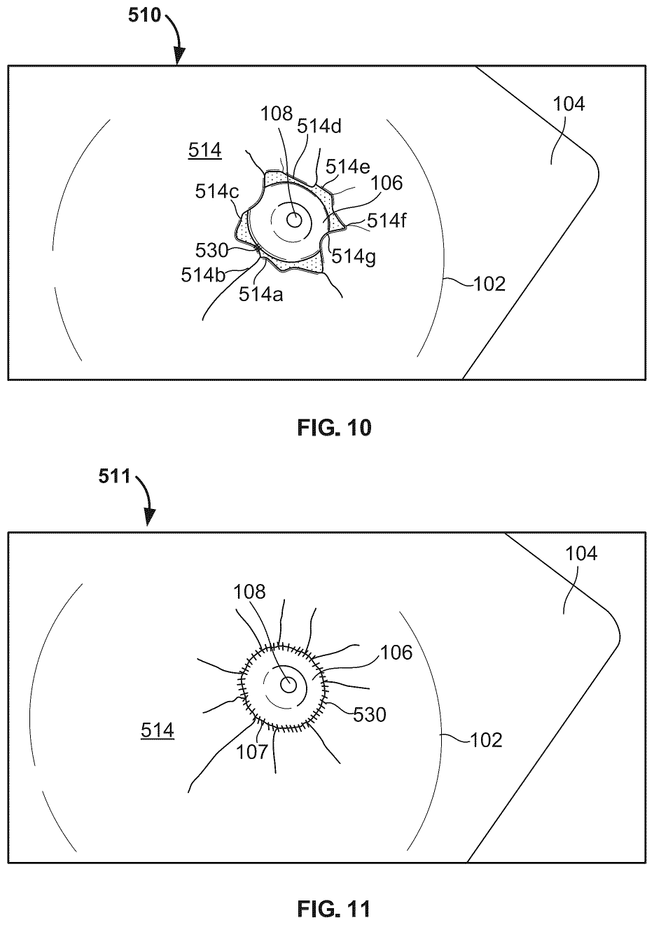

FIG. 10 shows the tissue retention system 100 at a step 510 the installation method where a user adjusted the portions 514a-g of the epidermis layer 514 surrounding the areola 106. The adjustments made by the user shape the portions 514a-g surrounding the areola 106 into a rounded shape that corresponds to the shape of the tissue retention system 100. The upper hooks 122 can be releasably connected to the portions 514a-g during steps 508-510 so that the user can continue to adjust the shape of the tissues surrounding the areola 106 before stitching the opening shut. The upper hooks 122 hold the epidermis layer 514 in tension and prevent the tissue from retracting away from the upper hooks 122. In some embodiments, the step 510 can include a user starting to install stitches 530 that extend through the epidermis layer 514 surrounding the areola 106. The stitches 530 can provide additional strength to hold the tissues in position along with the upper hooks 122.

FIG. 11 shows retention system 100 at a step 511 of the installation method where the epidermis layer 514 is closed around a circumference 107 of the areola 106. In some embodiments, the stitches 530 can extend around the circumference 107 to secure the rounded shape. The stitches 530 can be installed between the upper hooks 122 and an outer edge of the areola 106, thereby closing the surgical site with the tissue retention system 100 implanted in the breast 102. the upper hooks 122 hold the epidermis layer 514 in position during the closing of the epidermis layer 514.

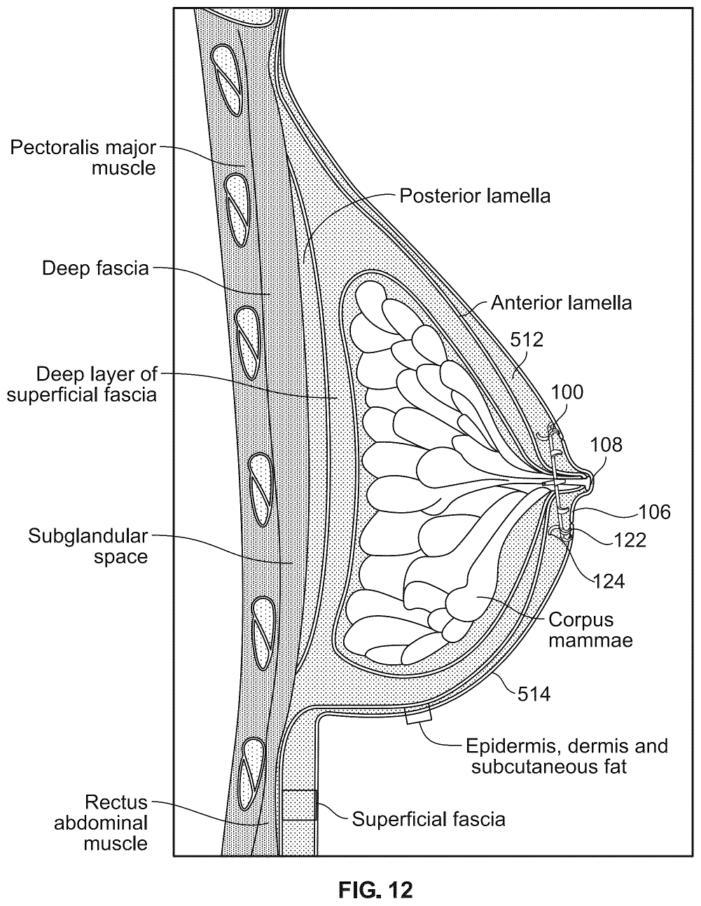

FIG. 12 shows a cross section of the breast with the tissue retention system installed 100 at the installation step 511 after the surgical site is closed. With the surgical site closed, the tissue retention system 100 is positioned posterior to the epidermis layer 514. The upper hooks 122 are positioned anterior to the lower hooks 124. The upper hooks 122 can connect to the posterior side of the epidermis layer 514 without penetrating through the anterior surface of the epidermis layer 514. The tissue retention system 100 biodegrades within the breast 102 over a period after the surgical site is closed. The tissue retention system 100 remains in position during the period of time to facilitate healing of the tissues in the desired shape.

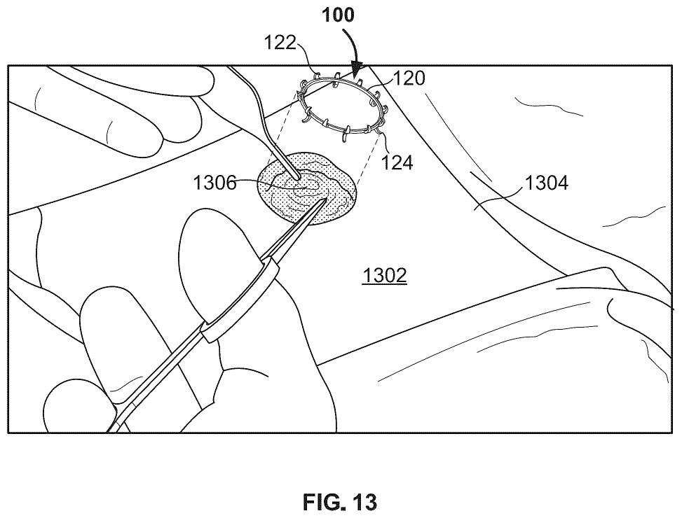

FIG. 13 shows the tissue retention system 100 that can be installed in an abdomen 1302 of a subject 1304. In some embodiments, the tissue retention system 100 can be implanted into a surgical opening 1310 in the abdomen 1302 to retain tissues around the surgical opening. In some embodiments, the system 100 can be installed during an abdominal cosmetic surgery. For example, the surgical opening 1310 can be created during a cosmetic surgical procedure such as a umbilicoplasty, abdominoplasty, tummy tuck, or other procedures. The system 100 can be installed around a belly button 1306 within the surgical opening 1310 to facilitate healing of the surgical opening 1310 around the belly button 1306 in a similar manner to the healing of the surgical opening 110 around the areola 106 discussed above.

The steps 505, 506, 508, 509, 510, and 511 can be implemented at the abdomen 1302 and surgical opening 1310. The lower hooks 124 can be inserted into a dermal layer of tissue at the abdomen 1302, and the upper hooks 122 can connect to a posterior side of an epidermis layer of tissue at the abdomen 1302.

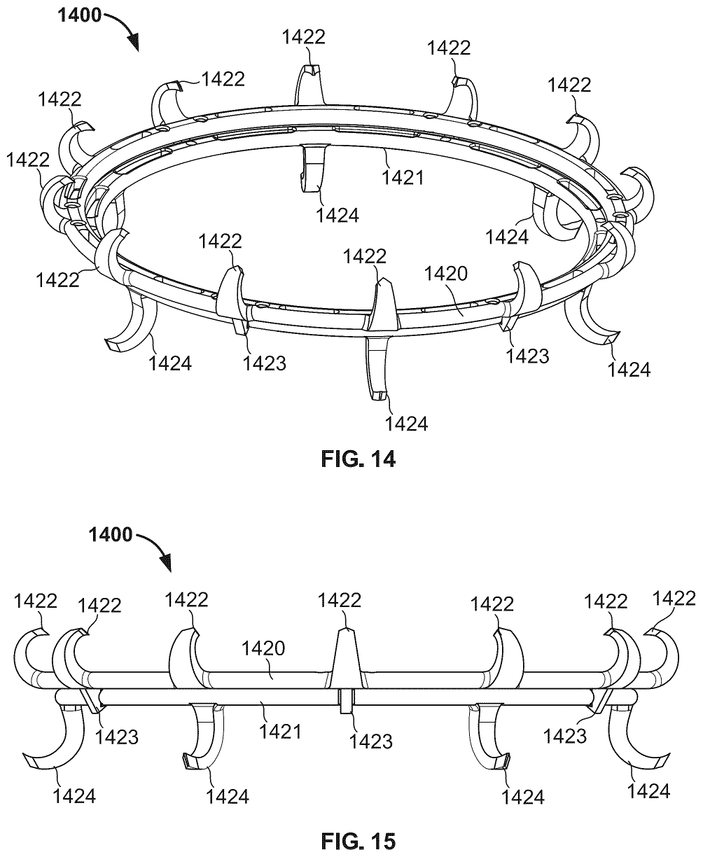

FIGS. 14 and 15 show a tissue retention system 1400. The tissue retention system 1400 can share features with the tissue retention system 100, including the upper hooks 1422 and lower hooks 1424 having similar shapes and positions as the upper hooks 122 and lower hooks 124. The tissue retention system 1400 can include bioresorbable materials included in the tissue retention system 100. The tissue retention system 1400 can include an upper frame 1420 and a lower frame 1421. The upper hooks 1422 can connect to the upper frame 1420 and the lower hooks 1424 can connect to the lower frame 1421. The upper frame 1420 can connect to the lower frame 1421 via frame hooks 1423 that extend from a bottom surface 1425 of the upper frame 1420. The frame hooks 1423 can facilitate attachment and detachment of the upper frame 1420 from the lower frame 1421. For example, the lower frame 1421 and lower hooks 1424 can be detached from the upper frame 1420 before installation. The lower frame 1421 and lower hooks 1424 can be installed in the subject and subsequently the upper frame 1420 can be attached to the lower frame 1421 by connecting the frame hooks 1423.

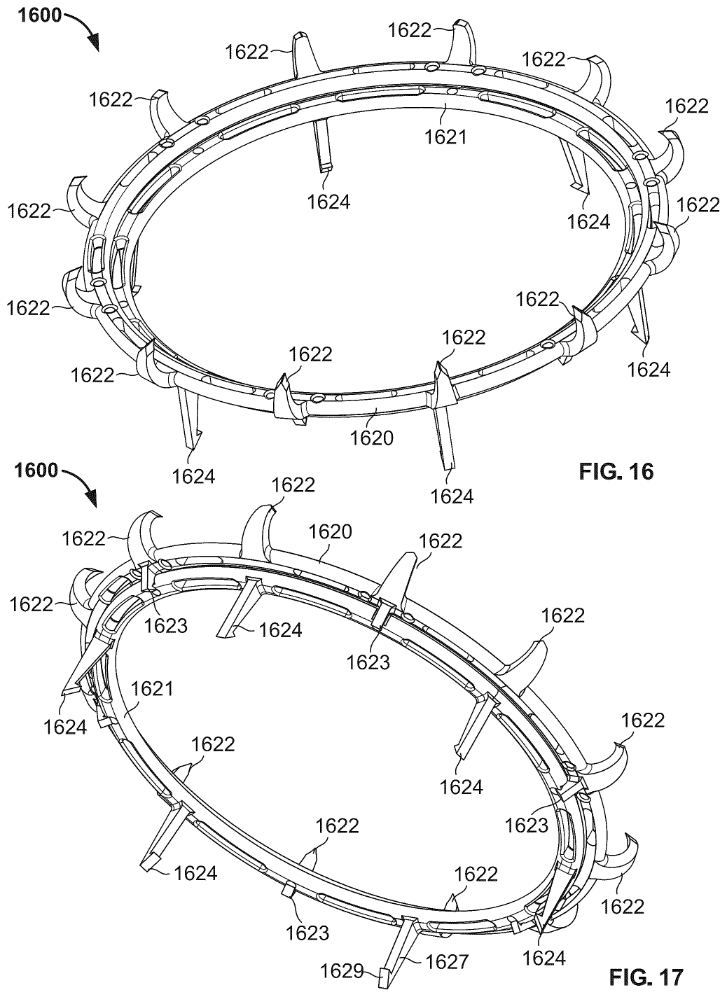

FIGS. 16 and 17 show a tissue retention system 1600. The tissue retention system 1600 can share features with the tissue retention systems 100 and 1400, including the upper hooks 1622 having similar shapes and positions as the upper hooks 122 and the upper hooks 1422. The lower hooks 1624 can differ in shape from the lower hooks 124 and can have a tapered profile that extends from a lower frame 1621. In some embodiments, the lower hooks 1624 have a post portion 1627 and a hooked end 1629. The tissue retention system 1600 can include bioresorbable materials included in the tissue retention system 100. The tissue retention system 1600 can include an upper frame 1620 and the lower frame 1621. The upper hooks 1622 can connect to the upper frame 1420 and the lower hooks 1624 can connect to the lower frame 1621. The upper frame 1620 can connect to the lower frame 1621 via frame hooks 1623 that share features with the frame hooks 1423.

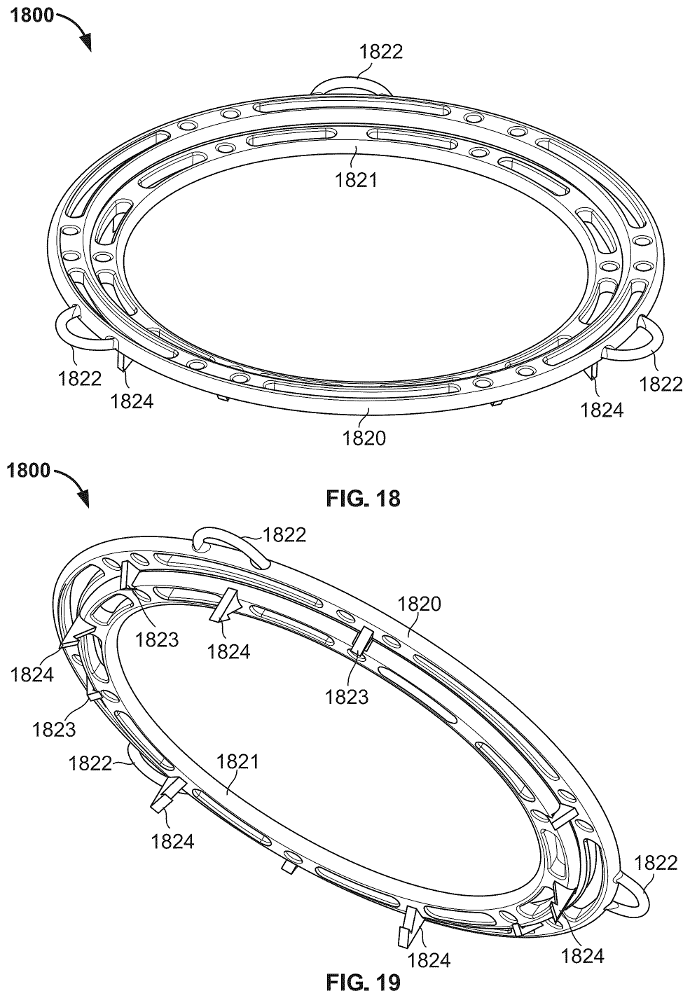

FIGS. 18 and 19 show a tissue retention system 1800. The tissue retention system 1800 can share features with the tissue retention system 1600, including the upper frame 1820 and the lower frame 1821 that can share some features including the frame hooks 1823 that connect the upper frame and lower frame together. The upper frame 1820 can include upper loops 1822 that extend from a lateral surface of the upper frame 1820. The upper loops can extend laterally and be configured such that stitches can be installed through the upper loops 1822 during installation of the system 1800. The system 1800 can also include lower hooks 1824 that share features with lower hooks 1624. In some embodiments, the lower hooks 1824 can be shorter in length than the lower hooks 1624.

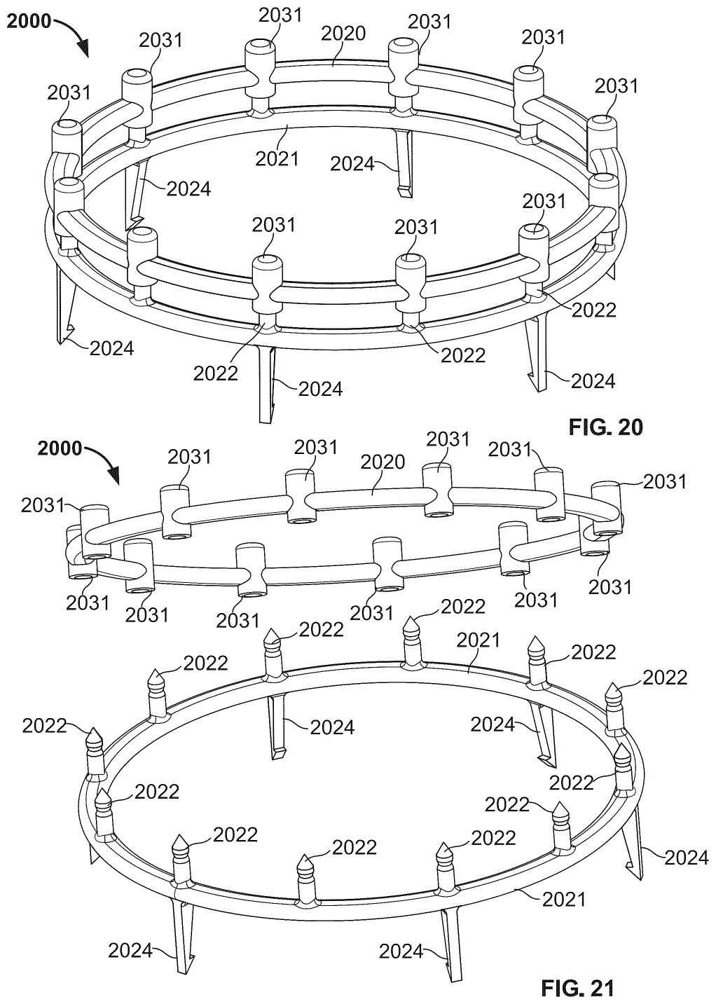

FIGS. 20 and 21 show a tissue retention system 2000. The tissue retention system 2000 can include an upper frame 2020 and a lower frame 2021. The upper frame 2020 can include a plurality of post receptacles 2031 spaced apart around the upper frame 2021. The lower frame 2021 can include a plurality of posts 2022 and lower hooks 2024. The plurality of posts 2022 are configured to align with the plurality of post receptacles 2031 to connect the upper frame 2020 and the lower frame 2021 together. The plurality of posts 2022 can include a spiked top surface and a barbed outer surface that facilitate a connection to tissue layers between the upper frame 2020 and the lower frame 2021.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

Accordingly, other implementations are within the scope of the following claims.

What is claimed is:

1. A tissue retention system, comprising:
a frame;
upper hooks extending from the frame that include a plurality of radially inward arcuate hooks such that a free end of each of the upper hooks is oriented directionally inward towards a central axis of the tissue retention system; and
lower hooks extending from the frame that include a plurality of radially outward arcuate hooks such that a free end of each of the lower hooks is oriented directionally outward away from the central axis of the tissue retention system.

2. The tissue retention system of claim 1, wherein the tissue retention system comprises a bioresorbable material.

3. The tissue retention system of claim 2, wherein the bioresorbable material is Poly lactic-co-glycolic acid (PLGA).

4. The tissue retention system of claim 1, wherein the frame is a ring.

5. The tissue retention system of claim 4, wherein the free end of each of the upper hooks is positioned above a longitudinal axis of the tissue retention system.

6. The tissue retention system of claim 5, wherein the free end of each of the lower hooks is positioned below a longitudinal axis of the tissue retention system.

7. The tissue retention system of claim 6, wherein an upper hook base of each upper hook connects each upper hook to a lateral surface of the frame.

8. The tissue retention system of claim 7, wherein a lower hook base of each lower hook connects each lower hook to a bottom surface of the frame.

9. The tissue retention system of claim 6, wherein the upper hooks have an upper hook height and the lower hooks have a lower hook height.

10. The tissue retention system of claim 9, wherein the upper hook height and the lower hook height are about equal.

11. The tissue retention system of claim 9, wherein the upper hook height is smaller than the lower hook height.

12. The tissue retention system of claim 1, wherein the system comprises a 2:1 ratio of upper hooks to lower hooks.

13. The tissue retention system of claim 1, wherein the frame has an inner diameter between 1.6 and 2 inches.

14. The tissue retention system of claim 1, wherein the frame has an outer diameter between 1.8 and 2.2 inches.

15. The tissue retention system of claim 1, comprising a system height that extends vertically between the free end of the upper hooks and the free end of the lower hooks.

16. The tissue retention system of claim 2, wherein the tissue retention system is configured for implantation in a breast of a subject.

17. The tissue retention system of claim 16, wherein the frame is configured to extend around a nipple of the breast.

18. A method of installing a tissue retention system, the method comprising:
preparing an installation site that includes a surgical opening in at least an epidermis layer of tissue of a breast of a subject;
aligning the tissue retention system around an areola of the breast;
inserting lower hooks of the tissue retention system into a dermal layer of tissue surrounding the areola;
positioning portions of the epidermis layer that surround the areola onto upper hooks of the tissue retention system; and
closing the epidermis layer by attaching the epidermis layer around a circumference of the areola between the upper hooks and an outer edge of the areola, thereby closing the surgical site with the tissue retention system implanted in the breast.

19. The method of claim 18, wherein closing the epidermis layer comprises installing stitches through the epidermis layer surrounding the areola and wherein the upper hooks hold the epidermis layer in position during the closing of the epidermis layer.

20. A tissue retention system for breast reconstruction, comprising:
a frame;
upper engagement tines extending from the frame that have free ends oriented directionally inward towards a central axis of the tissue retention system; and
lower engagement tines extending from the frame that have free ends oriented directionally outward away from the central axis of the tissue retention system.
